(19) United States
(12) Patent Application Publication
(10) Pub. No.: US 2006/0161200 A1
(43) Pub. Date: Jul. 20, 2006

(54) Apparatus and method for improving circulation of cerebral-spinal fluid

(76) Inventor: Afshin Al Fallah, Los Angeles, CA (US)

Correspondence Address: WOOD, NGO & EISENBERG, PLLC, 2121 Eisenhower Ave, Suite 200, Alexandria, VA, 22314, US

(21) Appl. No.: 11/332,289

(22) Filed: Jan. 17, 2006

Related U.S. Application Data
(60) Provisional application No. 60/644,561, filed on Jan. 19, 2005.

(51) Int. Cl. A61F 5/08

(52) U.S. Cl. 606/204.15

(57) ABSTRACT

An apparatus and method for improving cerebral spinal fluid (CSF) flow in a mammal having a skull made up of cranial sections. In one embodiment, the selective pressure is applied to a mammal's skull to induce movements in the cranial sections, wherein such movements cause improved CSF flow in the mammal. In a further embodiment, the CSF flow is improved by inducing movement in the vertebrae of the spine. In another embodiment, the invention is a cranial device, wherein the cranial device comprises a helmet and at least one reversibly inflatable member. In another embodiment, the invention is a spinal device for working on a patient's spine to improve CSF flow. In another embodiment, the invention is a combination of a cranial device and a spinal device.

### CROSS-REFERENCE TO RELATED APPLICATIONS

This application claims the benefit of priority from U.S. Provisional Patent Application Ser. No. 60/644,561, filed Jan. 19, 2005, the entire contents of which are incorporated by reference herein.

### STATEMENT REGARDING FEDERALLY SPONSORED RESEARCH OR DEVELOPMENT

Not Applicable.

### FIELD OF THE INVENTION

This invention relates to an apparatus and method for enhancing cerebral-spinal fluid flow by way of compression and decompression of identified pressure locations, and/or adjustment or movement of particular areas of the body.

### BACKGROUND OF THE INVENTION

It is thought that the majority of cells in the central nervous systems (CNS) of mammals obtain at least a portion of their nutrients via cerebral-spinal fluid (CSF). Using humans as an example, inside a person's cranium, there is a membrane called the dura mater that adheres to the bone. CSF is found between the dura mater and the next membrane. Similarly, in the spine, there is the bone of the vertebra, a layer of membrane within the vertebra and then CSF before the next layer of membrane. In the cranium, there are a total of three layers of membrane between the bones of the cranium and the brain. Likewise, in the spinal cord, there are three layers of membrane between the bone material of the vertebra and the CNS tissue of the spinal cord. It is thought that the CSF lubricates these membranes and by a process called diffusion delivers nutrients to the nerve tissue of the brain and spinal cord.

Various symptoms can occur if the circulation of CSF is interrupted or otherwise compromised. Persons with compromised circulation can experience certain symptoms such as tiredness, lack of mental focus, and lack of energy. Increasingly sedentary life styles associated with contemporary urban living can have a negative impact on normal circulation of CSF. Many occupations involve spending several hours sitting, for example, in front of computer screens. Jobs involving physical exertion are becoming less common in light of our increasing dependence on machines.

A review of the prior art follows.

U.S. Pat. No. 4,248,215, issued Feb. 3, 1981 to R. D. Feb. 3, 1981, describes an apparatus for relieving cranial tension comprising a head band having multiple diametrically disposed expandable compartments on the inner face of the head band, and an alternating pump for introducing and removing fluid under pressure for expansion and contraction of said compartments while in contact with the head.

U.S. Pat. No. 4,944,289, issued Jul. 31, 1990 to C. J. Matthews, describes a headache relieving headband. The headband includes an annular strip of material having an outwardly presented surface and an inwardly presented surface. A channel is defined by the inwardly presented surface and the outwardly presented surface and extends longitudinally along at least a portion of the annular strip of material. Pressure inducers are used to apply pressure simultaneously to pre-selected points on the cranium.

U.S. Pat. No. 5,792,174, issued Aug. 11, 1998 to R. Ioan, describes a natural headache reliever that uses acupressure points on the scalp and upper posterior neck. The Ioan '174 patent further describes a cap-like device having an outer membrane and an inner membrane for placing on a wearer's head. The two membranes meet and are attached at their margins, thus forming an inner potential space between the membranes. The inner membrane, that which will be closest to the scalp of the wearer, will contain a plurality of immobile protrusions (5) at a distance of between 10 and 20 millimeters apart. Direct pressure will be applied by the protrusions on different acupressure points on the scalp and upper posterior neck. A pump will be used to create the pressure by introducing air into the potential space between the inner and outer membranes. The wearer controls the duration and amount of pressure, thereby ensuring his or her comfort while alleviating the head pain.

None of the above inventions and patents, taken either singly or in combination, is seen to describe the instant invention as claimed.

### SUMMARY OF THE INVENTION

An apparatus and method for improving cerebral spinal fluid (CSF) flow in a mammal having a skull made up of cranial sections. In one embodiment, the selective pressure is applied to a mammal's skull to induce movements in the cranial sections, wherein such movements cause improved CSF flow in the mammal. In a further embodiment, the CSF flow is improved by inducing movement in the vertebrae of the spine. In another embodiment, the invention is a cranial device, wherein the cranial device comprises a helmet and at least one reversibly inflatable member. In another embodiment, the invention is a spinal device for working on a patient's spine to improve CSF flow. In another embodiment, the invention is a combination of a cranial device and a spinal device.

### BRIEF DESCRIPTION OF THE DRAWINGS

FIGS. 1A through 1C respectively show side, front and top partial cutaway cross-section views of a cranial device according to the present invention.

FIGS. 1D and 1E respectively show side views of an intra-oral device in inflated and deflated modes of operation.

FIGS. 2A through 2C shows a further embodiment of the cranial device shown in FIGS. 1A through 1C.

FIGS. 3A through 3D show different views of a further embodiment of the cranial device according to the present invention.

FIG. 4 shows a further embodiment of the cranial device according to the present invention.

FIG. 5 shows a further embodiment of the cranial device according to the present invention.

FIG. 6 shows a further embodiment of the cranial device according to the present invention.

FIGS. 7A and 7B show two views of a further embodiment of the cranial device according to the present invention.

FIG. 8 shows a further embodiment of the cranial device according to the present invention.

FIG. 9 shows a further embodiment of the cranial device according to the present invention.

FIG. 10 shows a further embodiment of the cranial device according to the present invention.

FIG. 11 shows a further embodiment of the cranial device according to the present invention.

FIG. 12 shows a further embodiment of the cranial device according to the present invention.

FIG. 13 shows a spinal device according to one embodiment of the present invention.

FIG. 14 shows a further spinal device according to one embodiment of the present invention.

FIG. 15 shows a further spinal device according to one embodiment of the present invention.

FIG. 16 shows a further spinal device according to one embodiment of the present invention.

FIG. 17 shows the cranial device of FIG. 3A attached to an external attachment member.

FIG. 18 shows the cranial device of FIG. 3A coupled to the spinal device of FIG. 13.

### DETAILED DESCRIPTION OF THE INVENTION

The invention is a method and system for enhancing cerebral-spinal fluid (CSF) flow by way of controlled compression and decompression cycles at selected locations such as cranium bones, and/or adjustment or movement of particular areas of the body, such as the spine.

The compression and decompression may be administered in synchrony with the natural rhythm of inhalation and exhalation. Alternatively, compression and decompression may be administered independently or without regard to natural breathing. However, it is preferred to synchronize the compression/decompression rhythm to a person's breathing rhythm to maximize, for example, movement of one or more of the cranial bones to facilitate the movement of the CSF into the venous system thereby reducing cranial pressure and in turn facilitating blood flow from the arterial system into the cells of the central nervous system (CNS).

The terms “person”, “user” and “patient” are hereinafter regarded as equivalent terms.

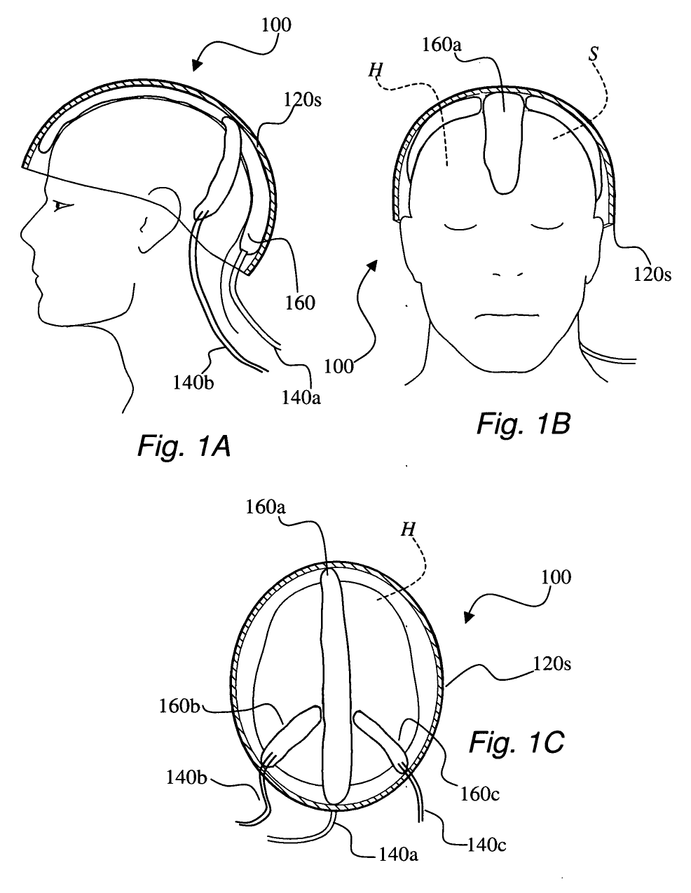

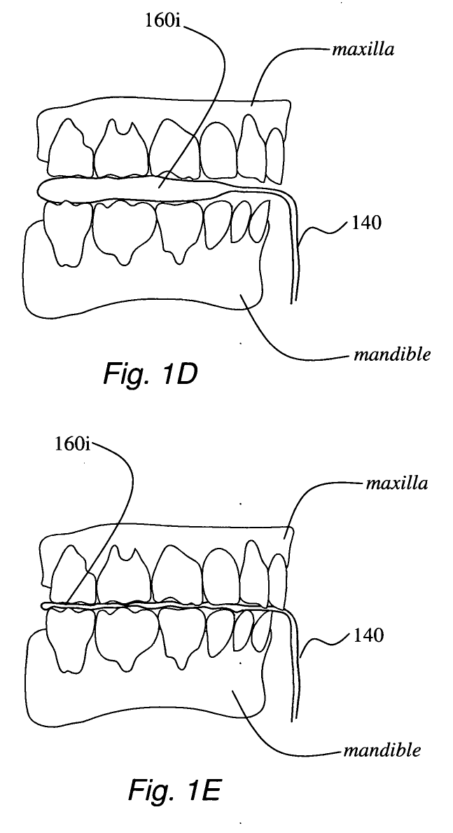

Referring to FIGS. 1A through 1C, an apparatus in the form of a cranial device 100, comprises a stretch-fit helmet 120s, at least one fluid delivery piping 140 (represented by, but not limited to, alpha-numeric labels 140a through 140c), and at least one reversibly inflatable member such as, but not limited to, at least one tube 160 (represented by, but not limited to, alpha-numeric labels 160a through 160c). The at least one tube 160 is used to apply pressure to selected parts of a mammal's head, such as a human head H. The fluid delivery piping 140 can deliver any suitable fluid such as a gas (such as air), a liquid (such as water), a plurality of solid particles (such as polystyrene beads), and any combination thereof. Hence, the term “fluid” is not limited to air or water, but is intended to cover any suitable material that can be delivered via piping 140 to the reversibly inflatable member such as, but not limited to, at least one tube 160.

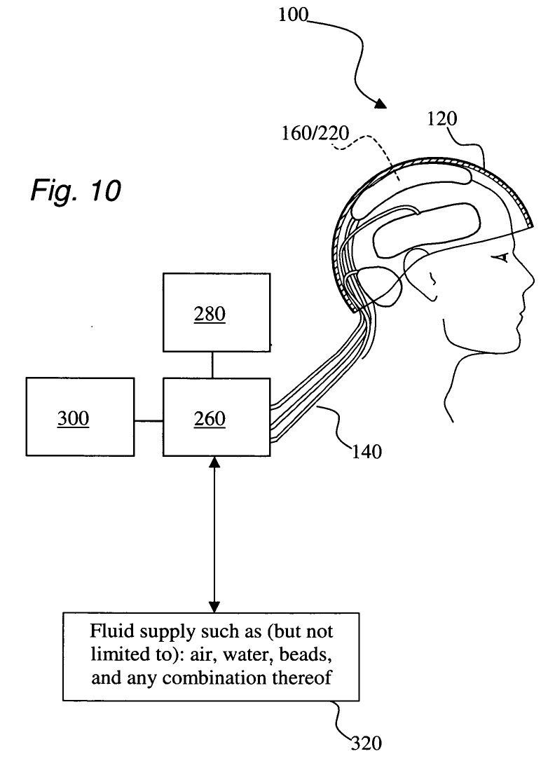

The at least one fluid delivery piping 140 can include a delivery and return fluid line. Alternatively, the at least one fluid delivery piping 140 acts as both a delivery and return line for reversibly inflating the at least one reversibly inflatable member. For example, the working fluid (such as air or water) could be pumped by a suitable pump into the at least one tube 160 and held in the at least one tube 160 for a predetermined time period. Once the predetermined time period has lapsed, the fluid drains via the at least one tube 140 from the at least one tube 160. A peristaltic pump could be used to pump the fluid, and then hold the fluid in place and then by reversing the pump, the fluid can be drained from the at least one tube 160. Alternatively, gravity and/or the at least one tube 160 can force the fluid out along the at least one pipe 140 (e.g., the at least one tube 160 can be made of an elastic polymer material such that upon expansion produces a responsive reaction force that acts on the fluid such the fluid is ejected along the piping 140 in absence of pump pressure. The pump element is shown, for example, in FIG. 10 (represented by numeric label “260”).

In typical operation, the at least one tube 160 is placed on a person's head H or skull S, and helmet 120s snuggly fitted over the at least one tube 160 such that the at least one tube 160 is sandwiched between the person's head H and the stretch-fit helmet 120s. The stretch-fit helmet 120s serves to hold in place the at least one tube 160. The at least one tube 160 is preferably filled and emptied of fluid (such as, but not limited to, air, water, beads, and any combination thereof) in rhythm with a person's natural breathing. For example, the at least one tube 160 can be pressurized during a user's exhalation and de-pressurized during inhalation or vice versa.

The at least one tube 160 (such as, but not limited to tubes 160a, 160b and 160c) can be positioned and/or sized to cover any suitable region of a person's cranium. For example, the at least one tube 160 can be made to cover the temporo-sephenoid and paraital sections.

The stretch-fit helmet 120s can be made of the same or similar polymer used in the manufacture of stretch-fit swimming caps that swimmers wear to minimize drag by covering head hair.

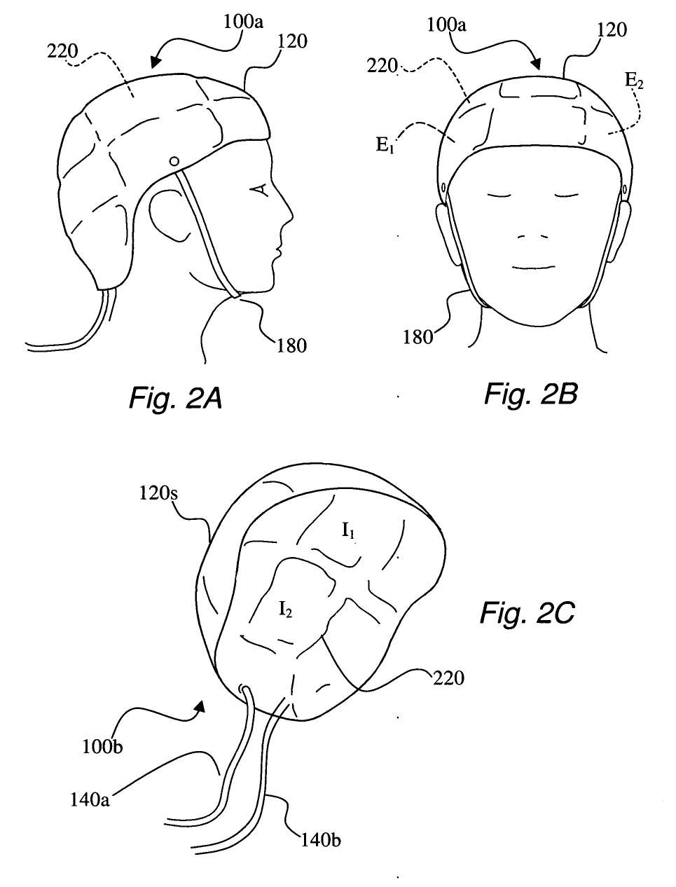

During operation, the at least one tube 140 is inflated and deflated in cycles to apply pressure to an area of a mammal's skull, such as a person's skull S. It is understood by those of ordinary skill in the art that a person's skull S is made up of cranial bones, which include the frontal, parietal, temporal, occipital, sphenoid, and ethmoid bones as described, for example, in the well-known Gray's Anatomy texts. It is also understood by those of ordinary skill in the art that the bones of the skull are joined together by sutures. For example, the coronal suture, which is found between the frontal and parietal bones; the lambdoidal suture, found between the parietal and the occipital bones; and the squamosal suture, found between the parietal and the temporal bone. The at least one tube 160 (or at least one pouch 220, see FIGS. 2A through 2C) is used to apply pressure to selected parts of the skull S to induce small movements between the cranial bones. The Applicant believes some movement occurs at the site of the cranial sutures thereby permitting some movement between the cranial bones. Specifically, upon inflating and deflating the at least one tube 160 and/or at least one pouch 220 and repeating the cycle, preferably in rhythm with a user's breathing, induces some movement in the sutures. Such movements help to induce increased flow in the CSF to benefit the cells found in the CNS.

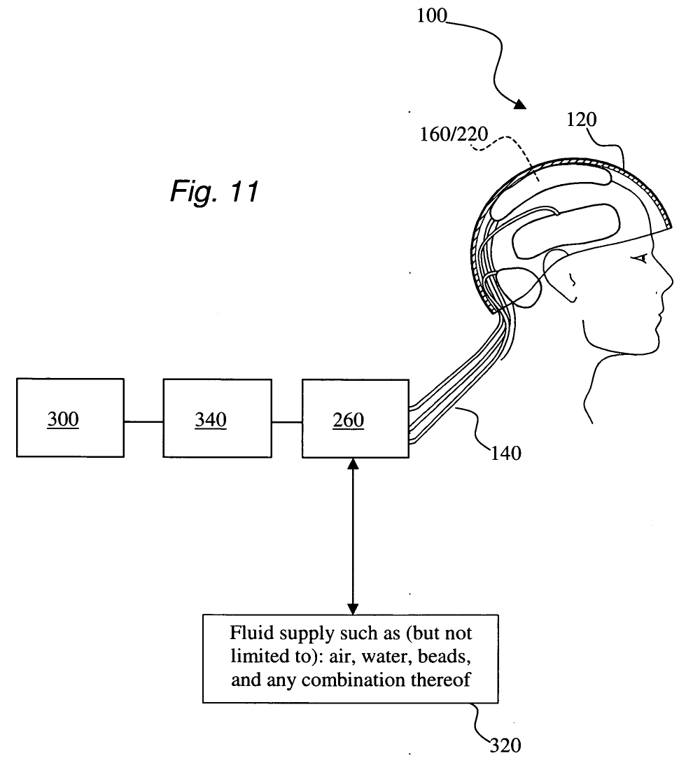

Still referring to FIGS. 1A through 1C, the at least one tube 160 can be designed to be reversibly filled with any suitable fluid (such as a gas, a liquid, a plurality of solid particles, and any combination thereof), such that when the helmet 120s is fitted to a user's head H, the at least one tube 160 is alternately filled and emptied of fluid thus applying pressure to an area of a person's skull S. The alternate filling and emptying of the at least one tube 160 can be synchronized with a person's breathing by simply setting the at least one tube 160 to fill and empty of fluid (such as air or water) at a predetermined rate (e.g., 15 cycles per minute of emptying and filling with air or water) and the patient or user voluntarily adjusting their breathing pattern to synchronize with the working of the cranial device 100, e.g. as air is pumped into the at least one tube 160, the user breathes in and exhales out air in time with the filling and emptying of the at least one tube 160. Alternatively, the user or patient can also use an on/off switch 340 (see FIG. 11) in series with a pump 260 to inflate and/or deflate the at least one tube 160 to help establish synchrony.

The at least one tube 160 can be arranged to exert pressure at selected areas on the user's head H and hence the user's skull S. By contacting the least one tubing 160 with the user's skull S at predetermined positions, cycles of pressure are applied to the user's cranium bones which in turn serve to aid the circulation of CSF about the user's body, thus providing extra nutrients, including oxygen, to the cells of the CNS.

The Applicant believes that cranium device 100 works by reducing or releasing stress across the cranial sutures located between adjacent cranium bones of the skull S. Modern convention is to believe that the cranium sutures serve to rigidly fuse adjacent cranium bones. However, it is thought that the at least one reversibly inflatable member of the invention, such as the at least one air or water inflatable tube 160 or at least one air or water inflatable pouch 220 (see, e.g., FIG. 2A) relieves undesirable stress and forces along cranium sutures located between adjacent cranium bones of the skull S. The Applicant believes that relieving stress in this manner in turn relieves distortions in the cranium bones that might otherwise cause unwanted compression zones in brain tissue beneath the skull S. More specifically, the Applicant believes that the cranial bones follow substantially opposite vectors of movement during cycles of compression and release of compression as provided by the at least one reversibly inflatable member of cranium device 100. However, it should be understood that any theory of how the cranial device 100 works is not intended to limit or reduce the scope of the cranial device 100 as defined in the attached claims.

At least one reversibly inflatable member can also be placed intra-orally. For example, at least one tube 160i can be placed intra-orally between the maxilla and mandible as shown in FIGS. 1D and 1E. The at least one tube 160i is inflated and deflated in the same manner as the at least one tube 160 or pouch 220 shown, for example, in FIG. 10. Specifically, the at least one tube 160i could be filled and deflated of working fluid (such as air, water or beads) via piping 140 connected to a pump 260 of FIG. 10. When the at least one tube 160i is inflated (as shown in FIG. 1D), as the user exhales, the user's mandible is brought forward (e.g., during exhalation), down (e.g., during inhalation), or down and forward. Alternatively, the at least one tube 160i is inflated and deflated independently of the patient's breathing rhythm.

Referring to FIGS. 2A and 2B, the cranial device 100 (actually represented by the alpha-numeric label 100a) comprises at least one reversibly inflatable member in the form of at least one pouch 220. The at least one pouch 220 is integrated into the helmet 120. The at least one pouch 220 is used to apply pressure to selected parts of a mammal's head, such as a person's head H. In typical operation, air or water is reversibly pumped to the at least one pouch 220 via flexible delivery piping 140 (represented by, but not limited to, alpha-numeric labels 140a and 140b). The at least one pouch 220 is filled and emptied of air or water in cycles, wherein the filling and emptying is preferably done in time with the user's breathing. A helmet strap 180 is used to hold the helmet 120 in place.

Referring to FIG. 2C, the cranial device 100 (actually represented by the alpha-numeric label 100b) comprises a stretch-fit helmet 120s. The stretch-fit helmet 120s negates or reduces the need for a helmet-restraining member such as strap 180 (see, e.g., FIG. 2A). A stretch-fit helmet 120s can be made, for example, of the same or similar polymer used in the manufacture of stretch-fit swimming caps that swimmers wear to maintain good hydrodynamics in swimming competitions. As stated, the stretch-fit helmet 120s can reduce or eliminate the requirement of a helmet-restraining member, such as, but not limited to, helmet strap 180.

Four of the at least one pouch 220 are labeled E₁, E₂, I₁ and I₂. The piping 140a of cranial device 100a can be used, for example, as follows: E₁ and E₂ are pressurized and de-pressurized when the user exhales and inhales, respectively; and piping 140b of cranial device 100a can be used, for example, as follows: I₁ and I₂ are pressurized and de-pressurized when the user inhales and exhales, respectively.

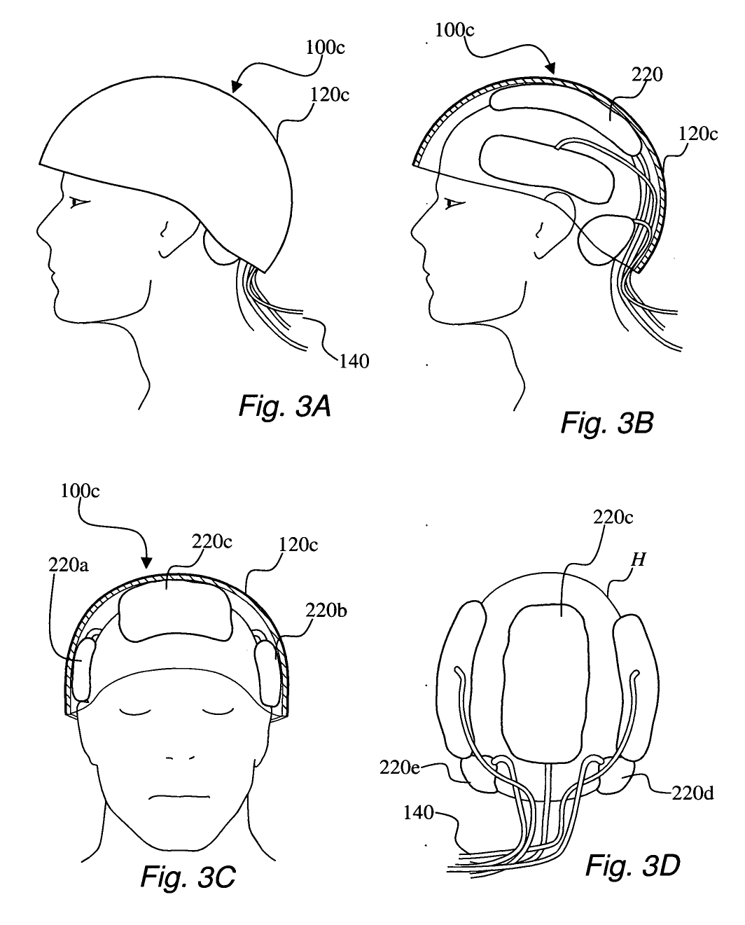

Referring to FIGS. 3A through 3D, the cranial device 100 (actually represented by the alpha-numeric label 100c) includes at least one reversibly inflatable member in the form of at least one pouch 220, rigid helmet 120c and delivery piping 140. The helmet 120c is held in place by, for example, pouches 220a and 220b, which upon inflation enable helmet 120c to resist forces from pouch 220c (see FIG. 3C). Alternatively, a helmet-restraining member can be used such as helmet strap 180 shown in FIG. 2A.

Pouch 220a may be used, for example, to apply on/off pressure to the frontal and paraital bones along the sagital and coronal sutures; and pouch 220b may be used, for example, to apply on/off pressure on the temporal, sephenoid areas; and pouches 220d and 220e can be used, for example, to apply on/off pressure to the mastoid areas of the patient's cranium.

It should be understood that the term “pressure” is meant to cover the on/off application of pressure by, for example, at least one tube 160 and/or at least one pouch 220.

The terms “pouch” and “pad” are regarded as equivalent terms herein. Pouches 220d and 220e are optionally positioned behind the patient's ears. It should be understood that the number of pouches and their exact location with respect to the helmet 120 (such as helmet 120s) is can vary.

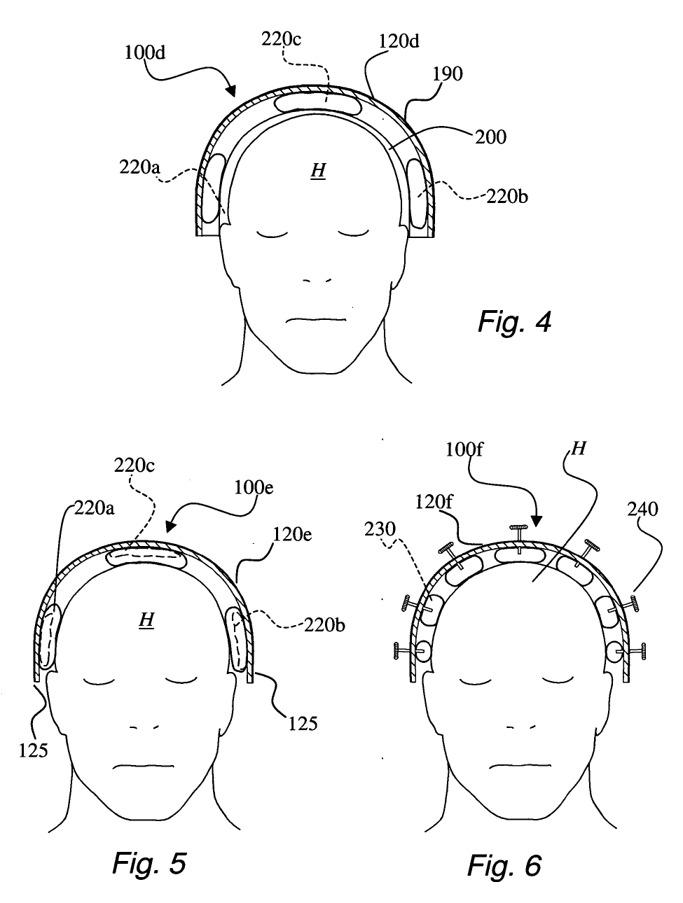

Referring to FIG. 4, the cranial device 100 (represented by alpha-numeric label 100d) includes a helmet 120d (shown in cross section). The helmet 120d has a rigid outer shell 190 and a stretch-fit inner layer 200, with at least one reversibly inflatable member sandwiched between the rigid outer shell 190 and the stretch fit inner layer 200. The at least one reversibly inflatable member is represented by, but not limited to, pouches 220a, 220b, and 220c. Pouches 220a and 220b may respectively apply pressure to the mastoid and temporal areas of the skull, and pouch 220a may apply pressure to the sagital suture.

Referring to FIG. 5, the cranial device 100 (represented by alpha-numeric label 100e) includes a helmet 120e (shown in cross-section). The helmet 120e is sized such that a user or wearer must lift back the lower rim 125 of the helmet 120e to get the helmet 120e onto the wearer's head H. Depending on the dimensions of the helmet 120e versus the dimensions of the wearer's head H, the pouches 220 (represented by, but not limited to, alpha-numeric labels 220a, 220b, and 220c) exert pressure on selected areas of the wearer's head H.

Referring to FIG. 6, the cranial device 100 (represented by alpha-numeric label 100f) includes a helmet 120f (shown in cross-section) and various adjusters 240. A plurality of solid pouches 230 is attached to the inside of the helmet 120f. The adjusters 240 are operably coupled to the pouches 230 such that if an adjuster 240 is turned (e.g., clockwise), the pouch 230 connected to that adjuster 240 moves away from the helmet 120f towards the wearer's head H, and in doing so, forces the helmet 120f to deform outward thereby inducing a counter force against the wearer's head H, while turning anti-clockwise causes the corresponding pouch 230 to move towards the interior of the helmet 120f thereby relieving pressure on the wearer's head H. By individually adjusting the adjusters 240, different degrees of force or pressure can be applied to selected cranium bones of the wearer's head H.

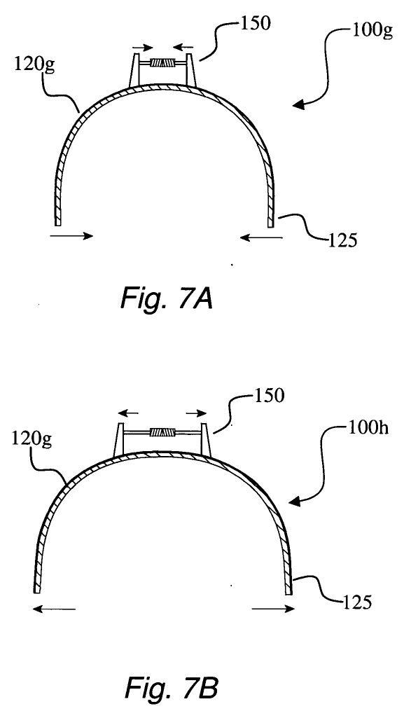

Referring to FIGS. 7A and 7B, the helmet 120 (actually represented by the alpha-numeric label 120g) is deformed in a similar fashion as with respect to helmet 120f except that the helmet 120g is deformed by means of a helmet shape adjuster device 150.

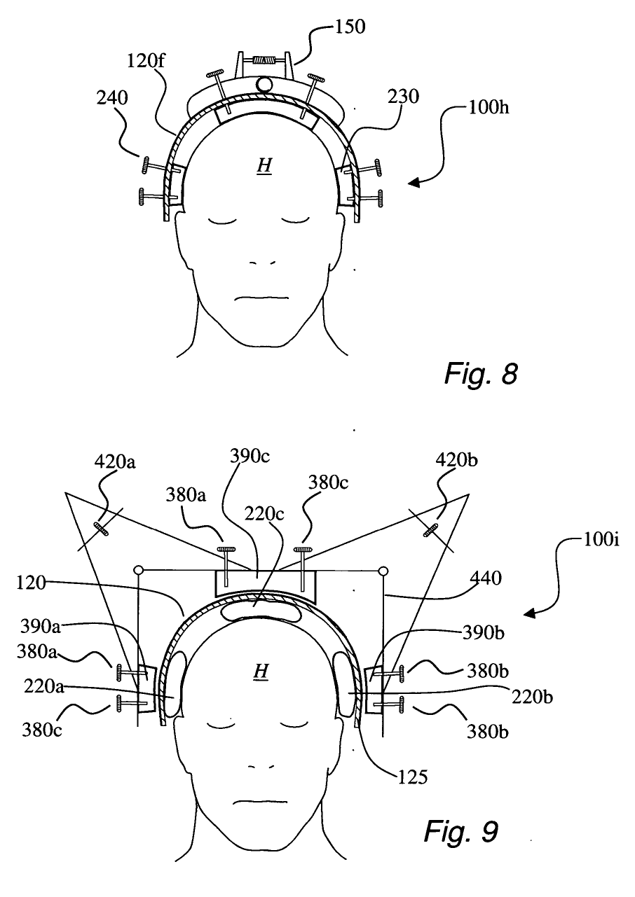

Referring to FIG. 8, which shows what could be described as a hybrid cranial device 100 (actually represented by alpha-numeric label “100h”). Specifically, the cranial device 100h incorporates the features shown in FIGS. 6 and 7A. More specifically, the cranial device 100h includes elements 150, 230, and 240.

Referring to FIG. 9, which shows a cranial device 100i inside an adjustable frame 440. Adjustment knobs 420a and 420b are used to adjust the sides of frame 440. More specifically, the knobs 420a and 420b can adjust the positions of pressure pads 390a, 390b, and 390c, which are attached to the sides of frame 440 by attachment knobs 380a, 380b, and 380c, respectively. The pressure pads 390a, 390b, and 390c in turn apply pressure or relieve pressure at various points on the helmet 120. For example, the rim 125 of helmet 120 can be moved inwards as required by applying pressure via pressure pads 390a and 390b. For example, by applying pressure via pads 390a and 390b, but relieving pressure with respect to pad 390c will cause the rim 125 to be pushed inward while allowing the top of the helmet 120 to expand outward.

It should be understood that while air or water can be used as the working fluid to alternately fill and empty the at least one reversibly inflatable member, any suitable working fluid can be used such as beads, e.g., beads of polystyrene or nylon. Any suitable dimension of beads can be used so long as the diameter of the beads does not impede their transfer into and out of the reversibly inflatable member or passage through piping 140. In addition, it should be understood that the at least one reversibly inflatable member is not limited to the at least one tube 160 but can be any suitable reversibly inflatable member such as, but not limited to, at least one pouch 220 as shown, for example, in FIG. 3B. The working fluid can be pumped around a closed circuit or via an open loop, e.g., air can be sourced from the surrounding air, pumped into the at least one reversibly inflatable member and thence back into the surrounding air. Alternatively, the working fluid can form part of a closed loop.

Referring to FIG. 10, the cranium device 100 is operably coupled to a pump 260, which in turn is operably coupled to a controller 280, and a power supply 300. The pump 260, under the control of the controller 300, supplies working fluid from a working fluid reservoir 320. The working fluid can be any suitable working fluid such as, but not limited to: air, water, beads, and any combination thereof. In FIG. 11, an on/off switch 340 in series with the power supply 300 is used to operate the pump 260.

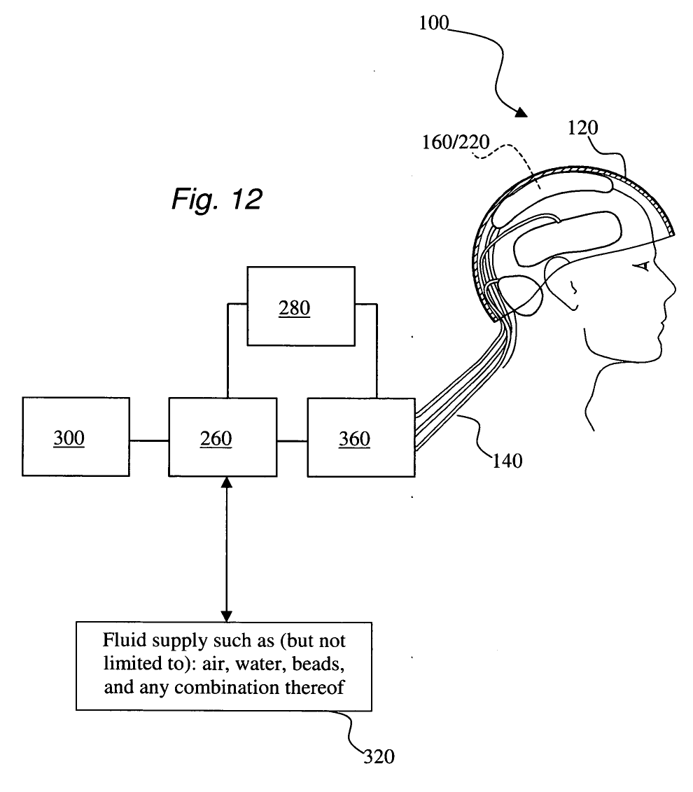

In FIG. 12, the controller 280 is operably coupled to the pump 260 and at least one temperature control 360; more than one temperature control can be used to maintain separate and different temperatures in the at least one reversibly inflatable member such as in each at least one tube 160 and at least one pouch 220, alone or in combination. The temperature control 360 is used to control the temperature of the working fluid, particularly if the working fluid has a high heat capacity and is a good conductor of heat energy and/or if the at least one reversibly inflation member has good head conductance and otherwise prone to making the user or patient uncomfortable absent a temperature control 360. The controller 280 includes a processor and sufficient memory to perform the logic steps necessary to operate the pump 260 and/or the temperature control 360. It should be understood that the component parts of the invention could be integrated. For example, the controller 280 can be integrated with the pump 260.

In addition to improving CSF flow in the cranium (i.e., between the dura mater and the next membrane) and the cranium-spine system, the cranial device 100 improves delivery of nutrients and oxygen to the CNS, and is expected to release stress along cranial sutures and thereby help reduce or prevent tension headaches and/or migraine headaches.

The at least one reversibly inflatable member (e.g., at least one tube 160 and/or pouch 220) and delivery piping 140 can be made out of any suitable material such as, but not limited to, polyethylene, PVC, and Santoprene™, and other polymers.

At first glance, it would appear that the pressure exerted by the at least one tube 160 and/or pouch 220 should be extensively tested using animal subjects. However, a little thought reveals that the amount of pressure should not exceed the level that becomes uncomfortable for the user or patient. Thus, the appropriate pressure is that which the user or patient can handle without feeling discomfort or pain. It is known for example, that a human diver can withstand considerable pressure to their bodies, including the bones of the skull. But it should not be forgotten that the principle under which the cranial device 100 operates depends on applying pressure designed to cause relative movement between the skull bones across the cranial sutures. Thus, pressure on one cranial bone absent pressure on adjacent cranial bones is most likely to achieve the desired movement without applying pressure to the entire skull.

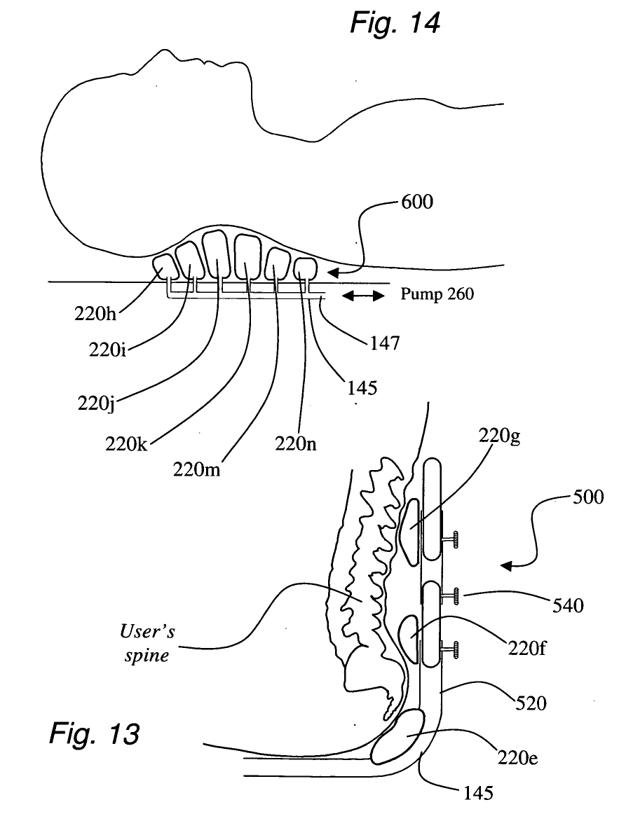

In another embodiment, the invention is a spinal device 500 (shown in FIG. 13) for causing movement in the vertebrae of the lower back to change the lordotic curves of the spine during inhalation and exhalation phases. The device 500 includes a support member 145 coupled to at least one inflatable pouch. In FIG. 13, the support member 145 is shown as an L-shaped telescopic tube 520; and the at least one reversibly inflatable pouch 220 is represented by, but not limited to, reversibly inflatable pouches 220e, 220f, and 220g located along the inside side of the L-shaped telescopic tube 520.

The L-shaped telescopic tube 520 can be extended and locked into position using any suitable locking means such as at least one locking knob 540. The reversibly inflatable pouches include pouches 220e, 220f, and 220g, which inflate and deflate independently of each other or in unison with each other. For example, pouches 220f and 220g inflates during exhalation by the patient while 220e deflates during the same exhalation by the patient. The pouches 220e, 220f, and 220g can be connected directly or indirectly to pump 260 and inflated with working fluid such as, but not limited to, air, liquid or beads. The support member 145 can be hollow and used as a conduit to deliver working fluid to/from the at least one reversibly inflatable pouch 220. The device 500 can work in either sitting mode or as a treatment with the patient in supine position. The term “reversibly inflatable pouches” and “reversibly inflatable cushions” are regarded here as equivalent terms.

In another embodiment, the invention is a spinal device 600 (shown in FIG. 14) for working on the vertebrae of the neck, and includes a support member 145 having a hollow interior 147. The support member 145 is used to support at least one reversibly inflatable pouch 220 (represented in FIG. 14 by pouches 220h, 220i, 220j, 220k, 220m, and 220n). The inflatable pouches 220h, 220i, 220j, 220k, 220m, and 220n are operably connected to a pump 260 (not shown), wherein working fluid is directed through the hollow interior 147 to the pump 260. Pouches increase in size from 220h to 220j, and backwards from 220n to 220k, with pouches 220j and 220k being about the same size. Thus, when these pouches are inflated, they support and move the neck vertebrae in a curved manner in keeping with the natural curve of the patient's neck and enhance the flow of CSF.

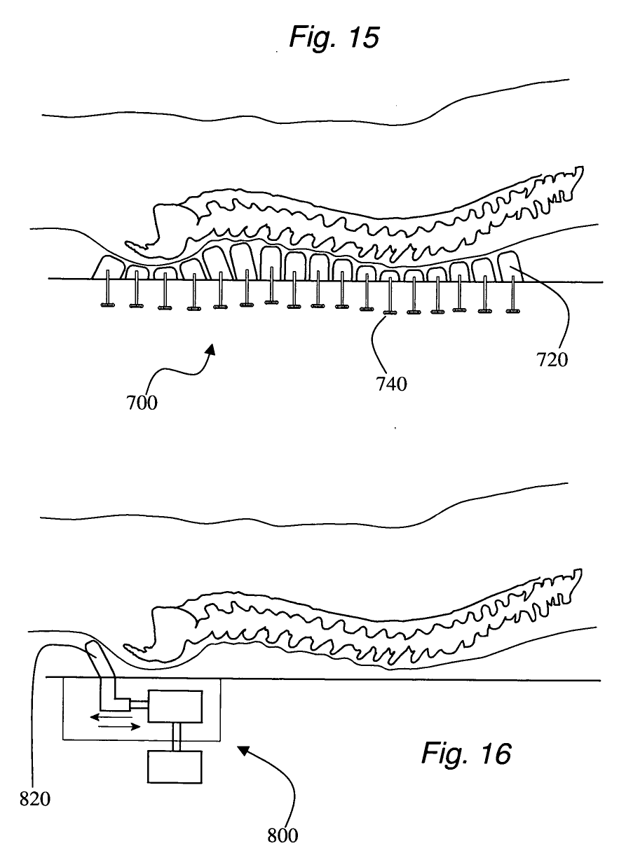

FIG. 15 shows a spinal device 700 for use on the lower and middle back regions of the spine. The device 700 comprises a plurality of substantially unyielding spinal elements 720, the height of which are adjustable by means of height adjusting knobs 740. A user or patient can position themselves on the device 700 as shown in FIG. 15. By moving their own body, the user can induce vector movements in their spine such that the flow of CSF is enhanced.

FIG. 16 shows another spinal device 800 comprising a moveable arm 820 for abutting against the lower part of the spine. The moveable arm 820 moves back and fore in a horizontal plain as shown. The device 800 takes advantage of a hypothesized dural sac called the sacral bulb, which acts as a reservoir for CSF. The arm 820 causes disturbance in the sacral bulb thus causing more CSF circulation.

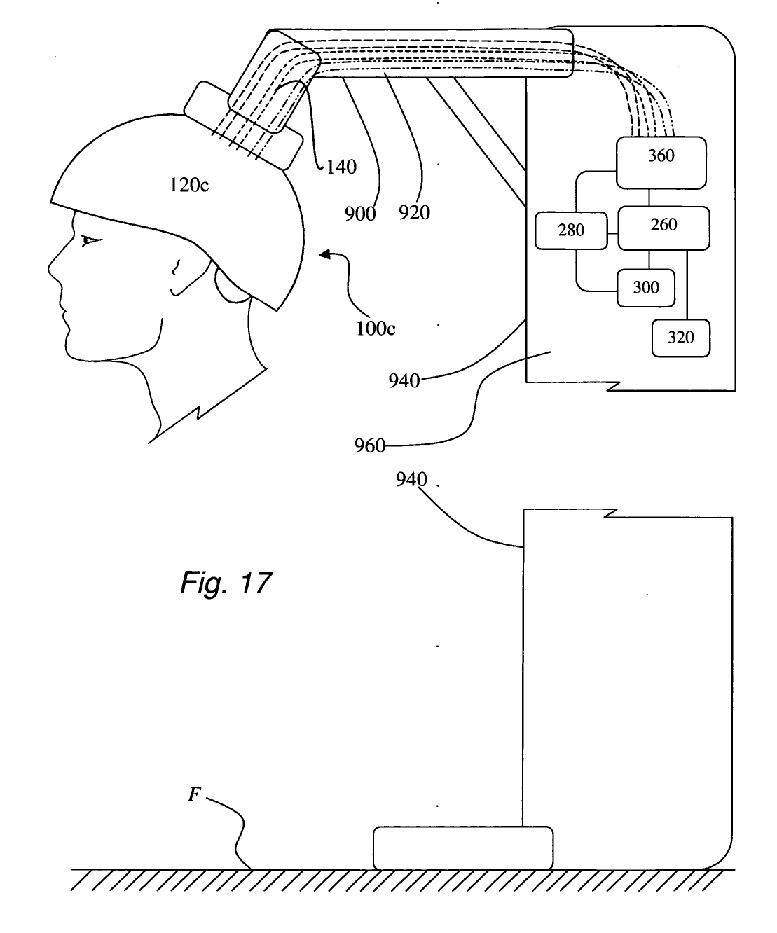

FIG. 17 shows the cranial device 100c of FIGS. 3A through 3D, connected to an external attachment member 900. The external attachment member 900 can include an essentially hollow interior 920. The external attachment member 900 can be further attached to a support frame 940. The support frame 940 can itself rest on a floor F. The external attachment member 900, and more particularly the interior 920, can act as a conduit for at least one piping 140. The support frame 940 can include a frame interior 960. The frame interior 960 can be used to house other elements such as, but not limited to: pump 260, temperature control 360, and power supply 300.

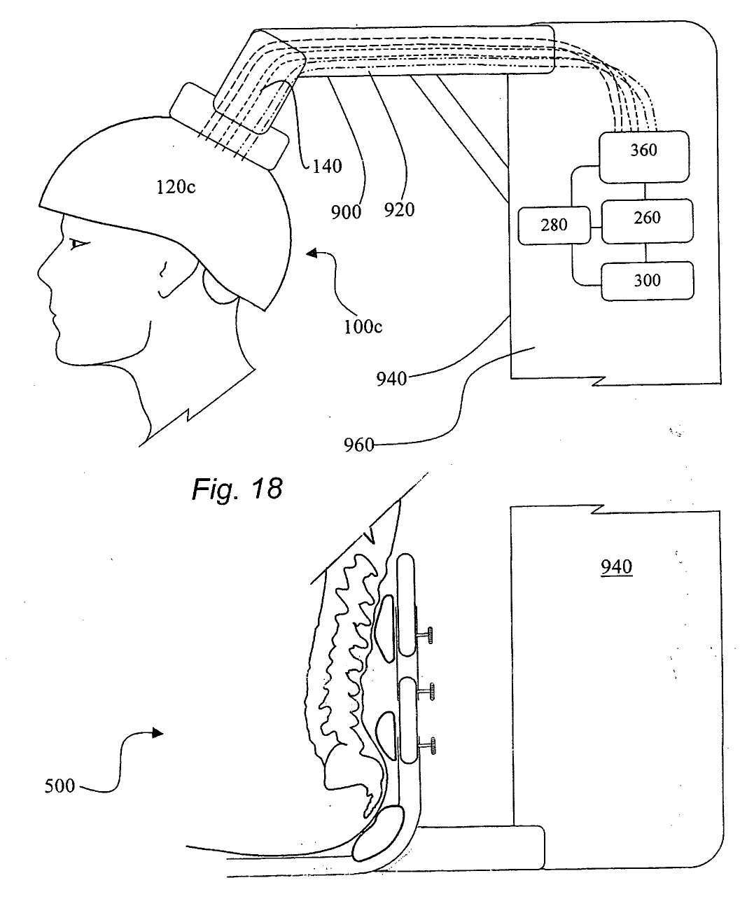

FIG. 18 shows the cranial device 100 (represented by, but not limited to, cranial device 100c) in combination with the spinal device 500 (shown in FIG. 13). The cranial device 100 can also be combined with spinal device 600 (shown in FIG. 14).

The above-mentioned devices and methods can operate singly or in combination as part of a system for improving circulation of CSF and blood flow to the central nervous system (CNS).

It is to be understood that the present invention is not limited to the embodiments described above or as shown in the attached figures, but encompasses any and all embodiments within the spirit of the invention.

1. A method for aiding cerebral spinal fluid (CSF) flow in a mammal having a skull made up of cranial sections, comprising the step of applying selective pressure to a mammal's skull to induce movements in the cranial sections, wherein such movements cause improved CSF flow in the mammal.

2. The method of claim 1, wherein the mammal is human.

3. The method of claim 1, wherein the mammal is selected from the group consisting of: a gorilla, a horse, a tiger, a bear, a lion, a zebra, an elephant, and a giraffe.

4. A method for improving cerebral spinal fluid (CSF) flow in a vertebrate, comprising the step of inducing movement in the vertebrae of the spine to improve CSF flow.

5. A cranial device for aiding cerebral spinal fluid (CSF) flow in a mammal having a skull made up of cranial sections, comprising: a helmet, and at least one reversibly inflatable member located inside the helmet.

6. The cranial device of claim 5, further comprising fluid delivery piping, wherein said fluid delivery piping is used to transfer working fluid to and from said at least one reversibly inflatable member.

7. The cranial device of claim 5, further comprising fluid delivery piping, wherein said fluid delivery piping is used to deliver a working fluid for inflating and deflating said at least one reversibly inflatable member, further wherein said working fluid is air.

8. The cranial device of claim 5, further comprising fluid delivery piping, wherein said fluid delivery piping is used to deliver a working fluid for inflating and deflating said at least one reversibly inflatable member, further wherein said working fluid is water.

9. The cranial device of claim 5, further comprising fluid delivery piping, wherein said fluid delivery piping is used to deliver a working fluid for inflating and deflating said at least one reversibly inflatable member, further wherein said working fluid is selected from the group consisting of: a gas, a liquid, a plurality of beads, and any combination thereof.

10. The cranial device of claim 5, wherein the helmet is a stretch-fit helmet.

11. The cranial device of claim 5, wherein said at least one reversibly inflatable member comprises at least one reversibly inflatable tube.

12. The cranial device of claim 5, wherein said at least one reversibly inflatable member comprises at least one reversibly inflatable pouch.

13. The cranial device of claim 5, further comprising fluid delivery piping, wherein said fluid delivery piping is operably connected to a pump, wherein said pump is used to deliver working fluid to said at least one reversibly inflatable member.

14. The cranial device of claim 5, further comprising fluid delivery piping, wherein said fluid delivery piping is operably connected to a pump, wherein said pump is used to deliver working fluid to said at least one reversibly inflatable member, and wherein said pump is controlled by a processor.

15. The cranial device of claim 5, further comprising fluid delivery piping, wherein said fluid delivery piping is operably connected to a pump, wherein said pump is used to deliver working fluid to said at least one reversibly inflatable member, and wherein said pump is controlled by an on/off switch.

16. The cranial device of claim 5, further comprising an external attachment member.

17. The cranial device of claim 5, further comprising an external attachment member and a support frame having a frame interior for

18. The cranial device of claim 5, further comprising an external attachment member and a support frame having a frame interior, wherein the frame interior houses a pump, a power supply, a controller, and a working fluid reservoir, and wherein said external attachment member acts as a conduit for at least one delivery piping for delivering working fluid to said at least one reversibly inflatable member.

19. A spinal device for manipulating a patient's spine, comprising a support member and at least one reversibly inflatable pouch.

20. The spinal of claim 19, wherein the spinal device is combined with a cranial device for manipulating a patient's cranium.
